(19) United States
(12) Patent Application Publication
(10) Pub. No.: US 2008/0135052 A1
(43) Pub. Date: Jun. 12, 2008

(54) RIGHT DOUBLE LUMEN ENDOBRONCHIAL TUBE

(76) Inventor: Jean BUSSIERES, Sainte-Foy (CA)

Correspondence Address: OGILVY RENAULT LLP, 1981 MCGILL COLLEGE AVENUE, SUITE 1600, MONTREAL, QC, H3A2Y3, omitted

(21) Appl. No.: 11/950,839

(22) Filed: Dec. 5, 2007

Related U.S. Application Data
(63) Continuation of application No. PCT/CA2006/000959, filed on Jun. 9, 2006.
(60) Provisional application No. 60/689,098, filed on Jun. 10, 2005.

(51) Int. Cl. A61M 16/00

(52) U.S. Cl. 128/207.15

(57) ABSTRACT

A right-sided double lumen tube (R-DLT) that is easy to position in a safe and effective manner so as to promote routine use thereof by all anesthesiologists. The R-DLT designed to facilitate the alignment of the lateral orifice thereof with the right upper lobe bronchus. The lateral orifice having an angular width of at least 80 degrees of 360 degrees of the circumference of a tube of the R-DLT.

### RELATED APPLICATION(S)

This application is a continuation of International Patent Application No. PCT/CA2006/000959 filed on Jun. 9, 2006, which claims benefit of U.S. Patent Application No. 60/689,098 filed on Jun. 10, 2005, which are herein incorporated by reference.

### TECHNICAL FIELD

The invention relates generally to a right double lumen tube used in endobronchial intubation for thoracic surgery, and more particularly to an improved right double lumen tube facilitating the alignment of the lateral orifice thereof with the right upper lobe bronchus.

### BACKGROUND OF THE INVENTION

Anesthetic techniques for thoracic surgery, either pulmonary or oesophageal necessitate the execution of endobronchial intubation. A variety of artificial airway devices have been developed to permit isolation of the lungs and to facilitate one lung ventilation (OLV). Separation of the lungs prevents the spread of secretions, pus and blood from one lung to the other. OLV is a process in which one lung is ventilated assuring gas exchange while the other lung is isolated and collapsed.

The advent of video-assisted thoracoscopic surgery (VATS) has increased the use of endobronchial intubation. A fibreoptic bronchoscope (FOB) is used to place an endobronchial tube under direct vision thereby facilitating the task of positioning same. Despite accurate initial placement of an endobronchial tube, movement can occur during anesthesia and surgery, and repositioning can be difficult.

The endobronchial double lumen tube (DLT) is the most common artificial airway device used to allow separate ventilation of the lungs. The DLT was first introduced in the beginning of the 60's and was greatly improved in the 80's with the emergence of polyvinyl chloride (PVC) DLTs. There exists a right-sided version (R-DLT) and a left-sided version (L-DLT) of the endobronchial double lumen tube (DLT), each version may be used in OLV of each respective lung.

The R-DLT and the L-DLT are designed differently particularly because of the anatomic variation between the tracheal carina, which is the first bifurcation of the tracheobronchial tree that separates the right lung from the left. Since the right upper lobe bronchus occurs a shorter distance after the carina than the left upper lobe bronchus, most R-DLTs have a lateral ventilation slot, orifice or the like that must necessarily be aligned with the origin of the right upper lobe bronchus for ventilation thereof. L-DLTs do not have this feature because of the longer left main bronchus.

It is widely known in the practice of thoracic surgery that the R-DLT is more difficult to position than the L-DLT. The difficulty lies in properly aligning the lateral orifice of the R-DLT and in maintaining the alignment thereof throughout the entire surgery. More specifically, proper placement of the R-DLT must endure changes in the patient's position such as from a dorsal decubitus position to a lateral decubitus position. In a case where optimal lateral orifice placement of the R-DLT is not realized, there lays a risk of anomalous ventilation of the lung which could result in clinical repercussions such as right upper lobe atelectasis and secondary hypoxemia.

Many anesthesiologists have published journals documenting the high failure rate of positioning the R-DLT and the risks involved. One example is a journal titled Con: Right-Sided Double-Lumen Endotracheal Tubes should Not Be Routinely Used in Thoracic Surgery by Edmond Cohen, MD published in the Journal of Cardiothoracic and Vascular Anesthesia, 2002; 16:249-52, the content of which is hereby incorporated by reference. The author arrives at the conclusion that “right-sided DLTs should not be used routinely in thoracic surgery.” The R-DLT is criticized for being more difficult to position and to manage during postoperative ventilation, also for having a high incidence of right upper lobe obstruction, and for being twice as expensive as left-sided DLTs (page 251, para. 6).

Another example is a journal titled Margin of Safety in Positioning Modern Double-Lumen Endotracheal Tubes by Jonathan L. Benumof, Brian L. Partridge, Cairo Salvatierra, and John Keating published in Anesthesiology 1987; 67:729-38 the content of which is hereby incorporated by reference. The authors make a clinical practice recommendation that “since the average margin of safety in positioning left-sided tubes is much greater than the average margin of safety in positioning right-sided tubes, left-sided tubes should be used whenever possible” (page 737, para. 2). Also, the authors have the opinion that “right-sided tubes are designed as well as they can be, and the margin of safety in positioning right-sided tubes cannot be improved” (page 738, para. 1).

Accordingly, official recommendations state that it is preferable to utilise the L-DLT whenever possible. As a result, the R-DLT has been largely abandoned in regular practice of thoracic surgery. Thus, anesthesiologists have generally become less skilled at operating with a R-DLT because of a lack of practice. However, when an absolute need to use a R-DLT arises, which is approximately 1-2% of surgical cases, anesthesiologists end up performing a surgery that they are not comfortable with. Consequently, the likelihood of complications occurring increases which is detrimental to the patient. Therefore, avoiding a particular practice in fact negatively impacts the anesthesiologists and in turns their patients.

A controversy over the use of the R-DLT does however exist as multiple journals advocating the use of the R-DLT have also been published. It is arguable that all anesthesiologists practicing in thoracic surgery must be adept in placing a R-DLT, and therefore the best way to achieve and maintain such a competence is through routine practice.

One example in which a view supporting the use of the R-DLT is expressed is in the journal titled Pro: Right-Sided Double-Lumen Endobroncheal Tubes Should Be Routinely Used In Thoracic Surgery by Javier H. Campos, MD, and Mark N. Gomez, MD published in the Journal of Cardiothoracic and Vascular Anesthesia, 2002; 16:246-8 the content of which is hereby incorporated by reference. The authors of the journal state that “the right-sided DLT is safe and efficacious when compared with either a single lumen tube with enclosed bronchial blocker or a left-sided DLT” (page 246, para. 4).

Another example is the journal titled Improving the Design and Function of Double-Lumen Tubes by Jonathan L. Benumof, MD published in the Journal of Cardiothoracic and Vascular Anesthesia, 1998; 2:729-33, the content of which is hereby incorporated by reference. The author proposes right-sided double-lumen tube design changes that may diminish the risk of right upper lobe obstruction. One of the principal changes suggested is to increase the length of the lateral ventilation slot of the R-DLT. Specifically, the author recommends having an approximately 20 mm long slot to communicate with an 11 mm diameter of the right upper lobe bronchus. Thus, in the event that the R-DLT moves in the axial direction of the right main bronchus subsequent to initial placement, the slot will still optimally ventilate the right upper lobe bronchus for a displacement of less than 4.5 mm in either direction.

A further example is titled Is it possible to Improve the Shape of the Right Double-Lumen Endobronchial Tubes? By F J. Mercier and M. Fischler published in the Journal of Cardiothoracic and Vascular Anesthesia 1995; 9:236, the content of which is hereby incorporated by reference. The authors have proposed to the manufacturers as a simplification in the use of right DLTs to remove the distal part of the tube comprising the lateral slot and keeping only the infero-internal part distal to the bronchial balloon.

Evidently, there is a need to render the R-DLT easy to position in a safe and effective manner so as to promote routine use thereof by all anesthesiologists. Although suggestions have been made concerning modifications to the R-DLT, none have proven to be fruitful in maintaining the lateral orifice optimally positioned with respect to the right upper lobe bronchus. Greatly, this is because up until now, the underlying problem behind the failing attempts at positioning the R-DLT has not been identified. Therefore, a hypothesis on the origination of the problem, based on the following clinical observations, is proposed.

### SUMMARY OF THE INVENTION

It is therefore an object of this invention to provide an R-DLT that is easy to position in a safe and effective manner so as to promote routine use thereof by all anesthesiologists

Another object of this invention is to provide an R-DLT designed to facilitate the alignment of the lateral orifice thereof with the right upper lobe bronchus.

It has been determined based on a number of clinical experiences involving the use of a R-DLT and the aid of a bronchoscope, that in order to optimally position the lateral orifice in regards to the right upper lobe bronchus it is often necessary to apply a rotation or torsion to the tube. The aforementioned rotation can be described relative to a coronal plane cutting the right main bronchus into anterior and posterior portions, the plane defining the zero degree point on the right main bronchus.

Most often in clinical practice, the R-DLT was rotated in the counter-clockwise direction causing an anterior displacement of the lateral orifice. However, in some cases the R-DLT was rotated in the clockwise direction to obtain a posterior displacement of the lateral orifice. It was observed that rotating the R-DLT facilitated the alignment of the lateral orifice with the right upper lobe bronchus. However, the rotation of the R-DLT would not persist due to the tube being made out of PVC, thus after a 15 to 20 minute period the lateral orifice would loose its optimal alignment.

As a result of the above-described observations, it has been discovered that the core problem arises from anatomical misconceptions. In fact, the origin of the right upper lobe bronchus is not always at zero degrees as it is commonly assumed to be but may vary by several degrees in the anterior or posterior direction.

Accordingly, an improved R-DLT addressing the above-described problem has been developed.

In one aspect, the present invention provides a right-sided double lumen tube for lung isolation, comprising a first and a second tube, each having a circumference defined by 360 degrees and a length defining a proximal and a distal end, the first and second tubes each defining a lumen at the respective distal end and being at least partially attached together along a portion of each of the first and second tube lengths, a tracheal cuff enveloping the first and second tubes and having a first inflation port and a first inflation catheter for fluid communication, the tracheal cuff being inflatable and deflatable by adding and removing fluid, respectively, to the tracheal cuff through the first inflation port and the first inflation catheter, a bronchial cuff enveloping the first tube proximal the distal end thereof and having a second inflation port and a second inflation catheter for fluid communication, the bronchial cuff being inflatable and deflatable by adding and removing fluid, respectively, to the bronchial cuff through the second inflation port and the second inflation catheter, and a lateral orifice defined about the circumference of the first tube proximal the lumen thereof and adapted to face a right upper lobe bronchus of a patient, the lateral orifice defining at least an angle of 80 degrees of 360 degrees of the circumference of the first tube and at most an angle permitting the first tube to retain its structural integrity, the lateral orifice facilitating alignment of the right-sided double lumen tube with the right upper lobe bronchus in various angular positions relative to a right main bronchus of the patient.

In another aspect, the present invention provides a right-sided double lumen tube comprising at least one endobronchial tube adapted for insertion in a right main bronchus of a patient, the tube having a circumference defined by 360 degrees and a length defining a proximal and a distal end, the tube defining a lumen at the distal end and a lateral orifice defined about the circumference proximal the lumen, the lateral orifice being oversized relative to a right upper lobe bronchus of the patient to an extent accommodating encountered anatomical angular deviations of the right upper lobe bronchus in anterior and posterior directions relative to the right main bronchus.

In a further aspect, the present invention provides a right-sided double lumen tube comprising at least one tube having a circumference and an axis, the tube having a length defining a proximal tracheal end and a distal bronchial end for communicating fluid therebetween, the tube defining a lateral orifice about the circumference at the distal bronchial end, the lateral orifice adapted to face a right upper lobe bronchus of a patient and to optimize fluid communication therewith, the lateral orifice having an angular width of at least approximately 20% of the circumference of the tube and at most the angular width permitting the tube to retain its structural integrity, the lateral orifice having an axial length at least equal to a diameter of the right upper lobe bronchus.

In accordance with a still further general aspect of the present invention, there is provided an endobronchial tube for insertion in a right main bronchus of a patient, comprising a tube body defining a lateral orifice adapted for fluid communication with a right upper lobe bronchus, the tube body being insertable in the right main bronchus in a first position in which the lateral orifice is generally facing the right upper lobe bronchus, the lateral orifice being sized independently of the right upper lobe bronchus position relative to the right main bronchus to accommodate, when the tube body is in the first position, anterior and posterior deviations of the right upper lobe bronchus relative to a coronal plane separating the patient's anatomy into ventral and dorsal portions.

Further details of these and other aspects of the present invention will be apparent from the detailed description and figures included below.

### BRIEF DESCRIPTION OF THE DRAWINGS

Reference is now made to the accompanying figures depicting aspects of the present invention, in which:

FIG. 1 is a schematic view of the anatomy of the tracheobronchial tree;

FIG. 2 is a schematic view of a right-sided double lumen endobronchial tube;

FIG. 3 is perspective view of a lateral orifice of the right-sided double lumen endobronchial tube of FIG. 2 in accordance with a preferred embodiment of the present invention;

FIG. 4 is a perspective view of the right-sided double lumen endobronchial tube of FIG. 2, showing the position of the lateral orifice with respect to the right upper lobe bronchus of FIG. 1;

FIG. 5 is an axial view of a distal end of a right tube of the right-sided double lumen tube of FIG. 2;

FIG. 6 is a schematic view of a right-sided double lumen endobronchial tube positioned in a right main bronchus in accordance with the prior art; and

FIG. 7 is a schematic view of a right-sided double lumen endobronchial tube of FIG. 2, shown positioned in a right main bronchus.

### DETAILED DESCRIPTION OF THE PREFERRED EMBODIMENTS

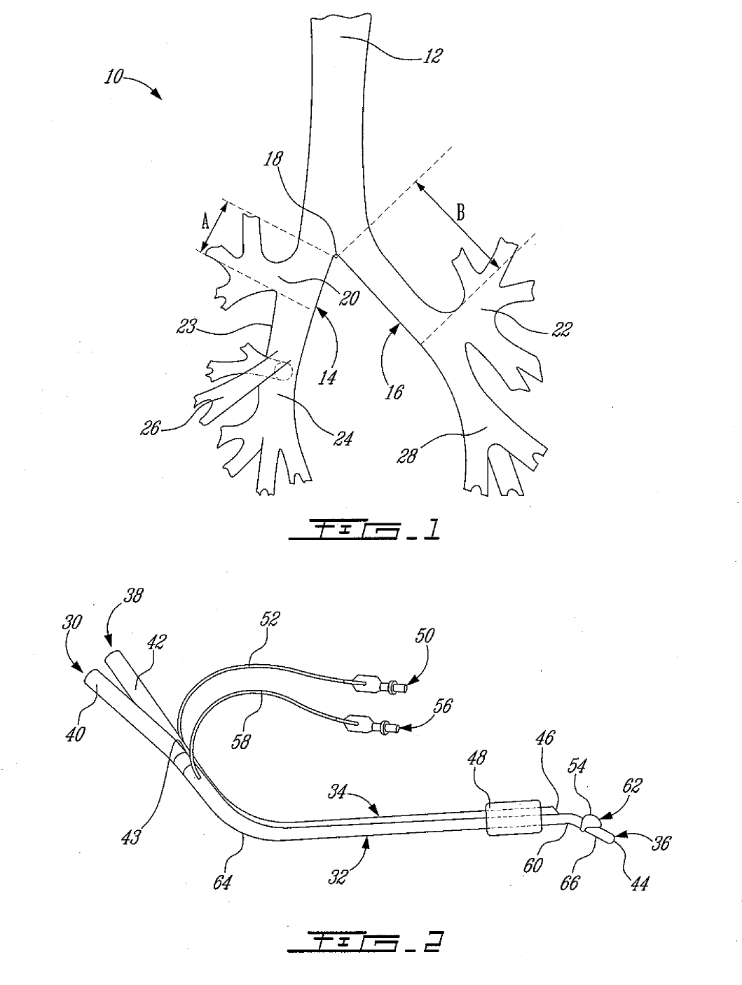

FIG. 1 illustrates the anatomy of the tracheobronchial tree represented by reference numeral 10. It can be seen that the tracheobronchial tree 10 comprises a trachea 12 that bifurcates into a right main bronchus 14 and a left main bronchus 16. The right main bronchus 14 branches off the trachea 12 at an approximate angle of 25 degrees and the left main bronchus 16 branches off at an approximate angle of 45 degrees. These are the major air passages from the trachea 12 to the lungs (not shown). Each lung is divided into upper and lower lobes, with the right lung also having a triangular division known as the middle lobe. The right lung is larger and heavier than the left lung, which is somewhat smaller in size because of the position of the heart. The main bronchi 14 and 16 enter each lung respectively and progressively branch off into more than 23 paired subdivisions. At every branching, the number of airways increases greatly. Thus, FIG. 1 illustrates a portion of the entire structure, resembling an upside-down branching tree, with the branches getting smaller and smaller as they get further from the trunk (trachea).

The junction point or keel-shaped anatomical part connecting the right and left main bronchi 14 and 16 is defined as the carina 18. The right main bronchus 14 ends at a first bifurcation with a right upper lobe bronchus (RULB) 20 and the left main bronchus ends at a first bifurcation with a left upper lobe bronchus (LULB) 22. It can be seen in FIG. 1 that the RULB 20 occurs a shorter distance A after the carina than distance B to the LULB 22. In addition, it can be seen that a right intermediate main bronchus 23 extends below the right main bronchus 14 and RULB 20 and further branches off into a right lower lobe bronchus 24 and a middle lobe bronchus 26. The left main bronchus 16 branches off into a left lower lobe bronchus 28.

It is accepted in the practice of anesthesia that anatomic variations of the RULB 20 position, due to anomalies in the development of the lungs, are common. Particularly, the axial position of the RULB 20 relative to the right main bronchus 14 can greatly vary from patient to patient. However, it is not yet recognized that the RULB 20 also greatly varies in its angular position relative to the right main bronchus 14, as such angular deviations are difficult to detect.

Now referring to FIG. 2, a basic design of a right-sided double lumen endobroncheal tube 30 (R-DLT) exemplifying one embodiment of the present invention is illustrated. Of course, it is to be understood that a multiplicity of R-DLTs are made by numerous manufacturers that vary from the basic pattern illustrated but that still fall within the scope of the present invention.

More specifically, the R-DLT exemplified in FIG. 2 is based on a model manufactured by Mallinckrodt Medical Inc. available in a variety of sizes. Presently available on the market are 7 different sizes of R-DLT's manufactured by various companies, 3 paediatric and 4 adult sizes. The paediatric sizes are 26-28-32 french and the adult sizes are 35-37-39-41 french in accordance with their outside diameter. Thus, the particulars of the present invention apply to any of the sizes for any of the models manufactured by different companies.

The R-DLT 30 shown in FIG. 2 comprises a first and a second tube 32 and 34 respectively, that are disposed adjacent to each other. Each tube 32, 34 has a length extending between a distal bronchial end 36 and a proximal tracheal end 38. The first and second tubes 32 and 34 are preferably partially attached together along a portion of their respective lengths between the distal bronchial end 36 and the proximal tracheal end 38. Generally, the first and second tubes 32 and 34 have a circular cross-section defining a circumference and an inner and outer diameter that vary depending on the size of the R-DLT 30 and the model thereof.

At the proximal tracheal end 38, the tubes 32 and 34 preferably branch into first branch section 40 and a second branch section 42 respectively. The bifurcation point is indicated by reference numeral 43, and is defined as the most proximal or uppermost location at which the first and second tubes 32, 34 are connected to each other. The bifurcation point 43 is the point above which the first and second branch sections 40, 42 begin, at the distal ends thereof.

The first and second branch sections 40 and 42 are adapted to be individually attached to a ventilation machine or respirator at the proximal tracheal end 38 thereof. The preferred materials used to form the tubes 32, 34 include polyvinyl chloride (PVC) and silicon, but one skilled in the art will appreciate that other surgical grade materials can be used, such as plastics and polymers.

In one embodiment, using first and second tubes 32 and 34 formed of PVC, the preferred way to fixedly attach sections of the tubes 32, 34 together is by forming or mouldings the two tubes 32, 34 to be integrally formed together. In another embodiment the first and second tubes 32 and 34 are preferably fixedly attached by way of fusion such that the fused sections have a D-shape cross-section and are fused along their respective straight edge cross-sectional portions. Still other connection designs exist such as by a chemical adhesive or by physical structures for maintaining a portion of the two tubes 32, 34 relative to each other.

The first and second tubes 32 and 34 comprise first and second lumens 44 and 46 respectively at the distal bronchial end 36. The first tube 32 is preferably longer than the second tube 34 such that the first lumen 44 extends further distally than the second lumen 46. The first lumen 44 is an endobronchial ventilation lumen for ventilating the middle lobe bronchus 26 and the right lower lobe bronchus 24. The second lumen 46 is a tracheal ventilation lumen for the left lung that opens above the carina 18, as opposed to the first lumen 44 that opens in the right main bronchus 14.

Adjacent the second lumen 46 proximal thereto, and attached to both the first and the second tubes 32 and 34, is a tracheal cuff 48. Particularly, the tracheal cuff 48 completely envelops the first and second tubes 32 and 34 of the R-DLT 30, such that, when inflated, it forms an air-tight seal within the trachea 12. The tracheal cuff 48 has an inflation port 50 and an inflation catheter 52 which extends through the wall of the second tube 34 preferably and connects the tracheal cuff 48 to the inflation port 50. By injecting fluid, such as air, into the tracheal cuff 48, a seal can be made to block the loss of positive pressure during ventilation.

Similarly, the first tube 32 comprises a bronchial cuff 54 proximal to the first lumen 44 that completely envelops the first tube 32 and forms an air-tight seal in the right main bronchus 14 when inflated. With the bronchial cuff 54 inflated, the right lung becomes isolated from the left lung. The bronchial cuff 54 also comprises an inflation port 56 and an inflation catheter 58 which extends through the wall of the first tube 32 respectively and connects the bronchial cuff 54 to the inflation port 56.

Notably, the bronchial cuff 54 is smaller is size than the tracheal cuff 48 due to the difference in diameter between the trachea 12 and the right main bronchus 14. It should be understood that many different cuff designs exist. In the case of the bronchial cuff 54, it is essential that the latter be designed to allow sealing and isolation of the right main bronchus 14 without occluding any of the upper lobe bronchi. Thus, as a first function, the bronchial cuff 54 ensures that the left lung remains collapsed during endobronchial intubation. As a second function, the bronchial cuff 54 helps maintain the R-DLT in proper axial position within the right main bronchus 14.

More specifically, the bronchial cuff 54 is located just below a bronchial curve 60 formed in the first tube 32. Hence the curved bronchial portion 62 of the first tube 32 is adapted to be deflected at the carina 18 so as to pass into the appropriate right main bronchus 14. The curved bronchial portion 62 is preferably deflected at an approximate angle of 25 degrees to match the natural right main bronchus 14 deflection.

In addition, both the first and second tubes 32 and 34 comprise an oropharyngeal curve 64. More specifically, the oropharyngeal curve 56 is formed near the bifurcation point 43 of the attached tubes 32, 34, curving in a direction opposite to that of the bronchial curve 60.

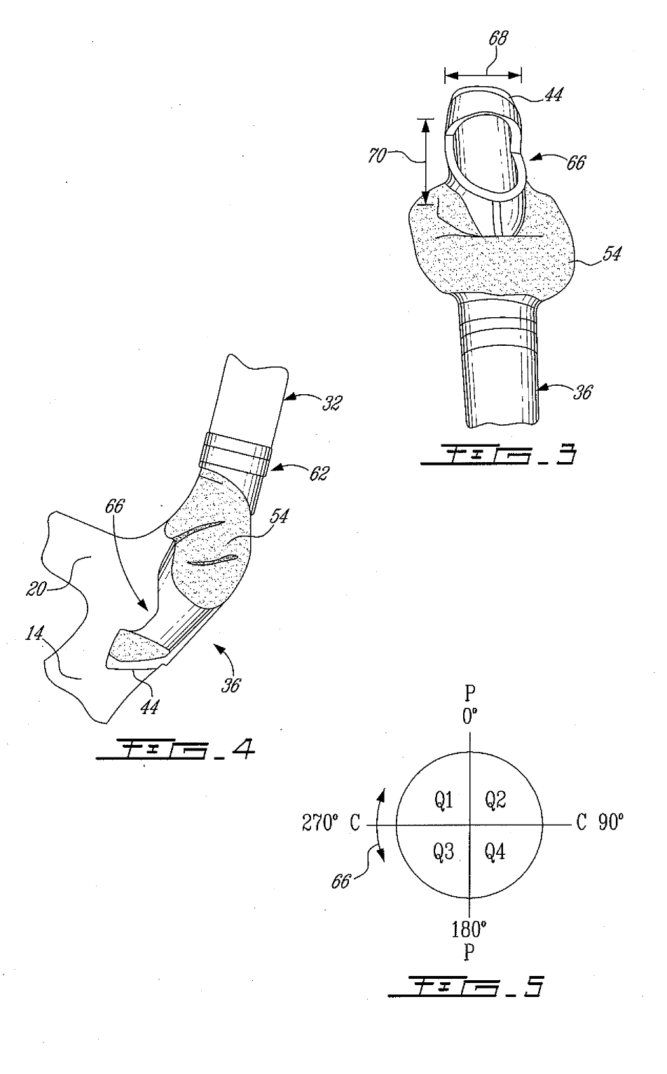

Referring now concurrently to FIGS. 2 to 4, the R-DLT 30, and more particularly the first tube 32 comprises a lateral orifice 66 defined in the wall thereof at the distal bronchial end 36. The lateral orifice 66 defines at least an angle of 80 degrees of the 360 degrees defining the circumference of the first tube 32. The lateral orifice 66 may be maximized up until but not including a point at which the structural integrity of the first tube 32 is compromised. The lateral orifice 66 preferably has an oval shape defining a transverse width and an axial length identified by numerals 68 and 70 respectively in FIG. 3.

Preferably, the lateral orifice 66 defines an angle of 180 degrees with respect to the 360 degrees defining the circumference of the first tube 32. The lateral orifice 66 is preferably located on the curved bronchial portion 62 of the first tube 32 just below the bronchial cuff 54 and proximal to the first lumen 44 and is adapted to face the RULB 20. The lateral orifice 66 is advantageously designed to facilitate alignment thereof with the RULB to provide ventilation thereto.

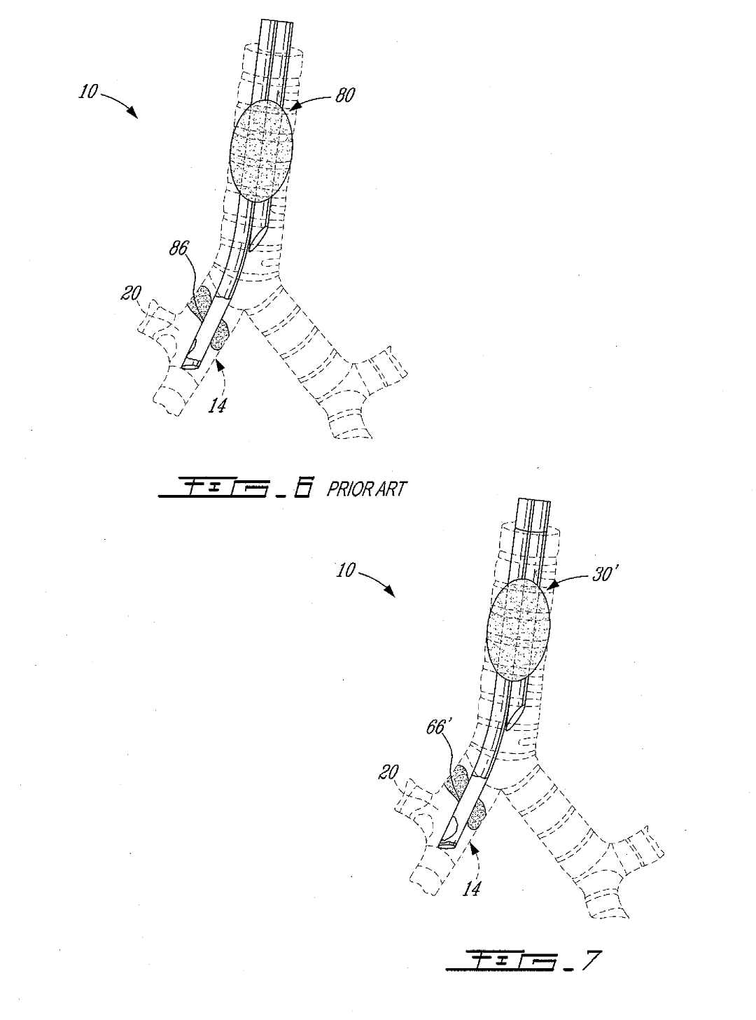

More specifically, FIGS. 4 and 5 illustrate the angular placement of the lateral orifice 66 with respect to the opening of the RULB 20. FIG. 5 illustrates an axial view of the first tube 32. The cross-section of the first tube 32 is divided into four equal quadrants Q1, Q2, Q3 and Q4 by two intersecting planes: a coronal plane C-C that cuts the first tube 32 into anterior and posterior halves (or top and bottom as shown in FIG. 5) and a transverse plane P-P that cuts the first tube in lateral halves. Hence, Q1 and Q2 define the anterior portion of the first tube 32 and Q3 and Q4 define the posterior portion. The anterior and posterior portions are described with respect to the ventral and dorsal portions of the human anatomy. Accordingly, the axis of the RULB 20 in a neutral position would be in line with plane C-C if the R-DLT were included in the right main bronchus 14. However, due to common anatomical deviations of the RULB's angular placement, the axis of the RULB is often angularly positioned several degrees above or below plane C-C. Therefore, the lateral orifice 66 that has an angle of 180 degrees of 360 degrees, i.e. the angular width 68 spans 80 degrees of the circumference of the first tube 32, is positioned in Q1 and Q3 facing laterally outward, centered about plane C-C.

The lateral orifice has an angular width 68 of at least approximately 20% of the circumference of the tube. However, it should be understood that a person skilled in the art would understand that the above percentage is not meant to limit the angular width 68 to a fixed value.

In the preferred embodiment, the lateral orifice 66 defines an angle of 180 degrees which spans from the 180 degree point to the 0 degree point indicated in FIG. 5. Thus, the lateral orifice preferably has an angular width 68 of 50% of the circumference of the tube. It should be appreciated that regardless of the angle defined by the lateral orifice 66, the latter is preferably always centered with respect to the axis of the RULB 20 when properly positioned in the right main bronchus 14.

Particularly, the lateral orifice 66 of the present invention is larger in the transverse direction of the first tube 32, as illustrated by width 68, than conventional slot designs. Enlarging the angular width 68 of conventional R-DLT lateral slots in the transverse direction by more than 100%, renders the task of positioning the R-DLT with respect to the RULB 20 less difficult. The enlarged lateral orifice 66 of the R-DLT 30 of the present invention can accommodate a variety of angular positions of the RULB 20 about the axis of the right main bronchus 14, as is commonly found in the human anatomy. As previously explained, the RULB 20 may have an anatomic position that is slightly anterior or posterior to a neutral position aligned with the coronal plane C-C (FIG. 5), cutting the right main bronchus 14 into anterior and posterior halves, that was previously believed to be the norm. Such a deviation is generally difficult for clinicians to detect. As the angular position of the RULB 20 may be several degrees forward or rearward from the neutral position, it is advantageous for the lateral orifice 66 to be wider than is necessarily required so as to accommodate the anatomic deviation of the RULB 20.

Notably, the length 70 of the lateral orifice 66 may be kept the same as conventional slot lengths or the lateral orifice 66 may also be lengthened in the axial direction of the first tube 32. The length 70 is preferably at least as long as the diameter of the RULB 20 as is best illustrated in FIG. 4. It should be understood the diameter of the RULB varies depending on the patient and therefore the length 70 of the lateral orifice 66 will also vary depending on the size of the model of R-DLT being used.

Due to the fact that the lateral orifice 66 extends a transverse width 68 ranging between 80 and 180 degrees about the circumference of the first tube 32, the alignment thereof with the RULB 20 is facilitated as the lateral orifice 66 is substantially larger than is required for ventilation. Thus, in the event that the R-DLT moves after initial placement, the lateral orifice 66 may still remain in alignment with the RULB 20 as its configuration allows for a degree of angular rotation of the R-DLT 30 without occluding the RULB 20. Therefore, the R-DLT 30 of the present invention address the need of providing a lateral orifice 66 that facilitates alignment with a RULB 20 that can vary in angular anatomical placement with respect to a neutral 0 degree position as was previously believed to be the norm. The R-DLT 30 encompassing the present invention is easy to position in a safe and effective manner thereby promoting routine use thereof by all anesthesiologists.

A study was designed to assess the impact of the aforementioned modification the lateral orifice with respect to conventionally sized slots on the success rate of the R-DLT's positioning.

### Method

Following Institutional Research Board (IRB) approval, 80 adult patients were randomly assigned to one of two groups to be intubated with an Original R-DLT (Bronco-Cath®, Malinckrodt, St-Louis, Mo., 63134) or Modified R-DLT. Referring to FIG. 6, the Original R-DLT identified by reference numeral 80 is shown inserted in the right main bronchus 14 of the tracheobronchial tree 10 with the lateral slot 86 facing the RULB 20. Referring to FIG. 7, the Modified R-DLT corresponds to the R-DLT shown in FIGS. 2 to 4 and identified by reference numeral 30′, is similarly shown inserted in the right main bronchus 14 of the tracheobronchial tree 10 with the lateral orifice 66′ facing the RULB 20.

The latter version of the R-DLT was modified manually by fixing the Original R-DLT to a customized jig and enlarging the lateral slot with a scalpel as permitted by the jig. The lateral orifice was enlarged by approximately 100%. This was done by increasing the angular width of the lateral orifice from the standard 66 degrees to 80 degrees so that the lateral orifice would occupy 50% of the endobronchial tube circumference. The length of the orifice was also augmented by a few millimetres distally. After modifying the tube, the integrity of the bronchial cuff was verified to see whether it was possible to cause a leak in the cuff. Furthermore, in order to endure reproducibility of these modifications and to minimize the risk of damaging the bronchial cuff, a template was used.

After induction of anaesthesia, the R-DLT was inserted. The position of the R-DLT was evaluated for each patient with assistance of a fibre optic bronchoscope, on 3 occasions:
1) Dorsal Decubitus Position (DDP) following optimal positioning of the R-DLT.
2) Immediately after lateral positioning (LP) of the patient.
3) Following optimal re-positioning of the R-DLT in LP.

The DLT's positions were categorized (1 to 4: 1=ideal and 4=worst) depending on the relative position of the right upper lobe opening (RULO) in regard to the RUL bronchus origin. More specifically, a complete visualization of the RULB origin was rated 1, partial visualization of the RULB origin was rated 2, visibility of the RULB origin only with minor rotation of the tube was rated 3, and absence of visualization of the RULB origin with minor rotation of the tube was rated 4. The Fisher exact test was performed to analyse categorical data. The results were considered significant with p-values≦0.05.

The objective of the study was demonstrate by fibre optic bronchoscope examination that the Modified R-DLT 30′ (FIGS. 2-4 and 7) more frequently maintained an adequate position after turning the patient in the lateral position and was also easier to reposition than the Original R-DLT 80 (FIG. 6).

RESULTS

| Score # 1 (ideal position) | | |
|---|---|---|
| Original R-DLT | Modified R-DLT | p value |

| | | | | | |
|---|---|---|---|---|---|
| DDP optimal | 35 | (88%) | 40 | (100%) | 0.0547 |
| LDP 1^{st} look | 8 | (20%) | 28 | (70%) | 0.0001 |
| LDP optimal | 23 | (58%) | 40 | (100%) | 0.0001 |

### Discussion

This preliminary study demonstrates that:
1) Initially, in DDP, Modified R-DLT seems easier to position in the ideal position
2) Following LP, the ideal position is more frequently maintained with the Modified R-DLT.
3) Finally, following LP, it is easier to re-obtain the ideal position with the Modified R-DLT.

The results illustrate that Modified R-DLT reduces the difficulty of optimally positioning the tube relative to the RULB angular position thereby improving the safety and effectiveness of using same. The configuration of the lateral orifice of the Modified R-DLT ensures optimal placement of the tube throughout the operation thereby minimizing the necessity of tube repositioning during the critical phase of a pulmonary procedure. This is advantageous as any tube manipulation during surgery is inconvenient, time-consuming, and potentially deleterious with respect to ventilation and gas exchange and may put the patient at risk of contamination or aspiration. The results of the above randomized trial clearly suggest the superiority of the Modified R-DLT when compared with the Original R-DLT to be optimally positioned for OLV to ensure optimal fluid communication.

In use for ventilating a lung of a patient, the R-DLT 30 of the present invention is passed through the trachea 12 and partially into the right main bronchus 14 of the patient. The lateral orifice 66 of the first tube 32 of the R-DLT 30 is optimally aligned with the RULB. Once the R-DLT 30 has been properly positioned the tracheal cuff 48 and bronchial cuff 54 are inflated to secure the R-DLT 30 in place and to isolate the lungs the first tube 32 at the proximal tracheal end 38 is connected to a respirator. The above-described method can include the use of a fibreoptic bronchoscope (FOB) to place the R-DLT 30 under direct vision thereby facilitating the task of positioning same.

The above description is meant to be exemplary only, and one skilled in the art will recognize that changes may be made to the embodiments described without department from the scope of the invention disclosed. For example, the lateral orifice may be provided in a variety of shapes so long as the transverse width thereof is at least equal to or greater than 80 degrees. Also, the lateral orifice of the R-DLT may be manufactured by many different methods. Still other modifications which fall within the scope of the present invention will be apparent to those skilled in the art, in light of a review of this disclosure, and such modifications are intended to fall within the appended claims.

1. A right-sided double lumen tube for lung isolation, comprising:
a first and a second tube, each having a circumference defined by 360 degrees and a length defining a proximal and a distal end, the first and second tubes each defining a lumen at the respective distal end and being at least partially attached together along a portion of each of the first and second tube lengths;
a tracheal cuff enveloping the first and second tubes having a first inflation port and a first inflation catheter for fluid communication, the tracheal cuff being inflatable and deflatable by adding and removing fluid, respectively, to the tracheal cuff through the first inflation port and the first inflation catheter;
a bronchial cuff enveloping the first tube proximal the distal end having a second inflation port and a second inflation catheter for fluid communication, the bronchial cuff being inflatable and deflatable by adding and removing fluid, respectively, to the bronchial cuff through the second inflation port and the second inflation catheter; and
a lateral orifice defined about the circumference of the first tube proximal the lumen thereof and adapted to face a right upper lobe bronchus of a patient, the lateral orifice defining at least an angle of 80 degrees of 360 degrees of the circumference of the first tube and at most an angle permitting the first tube to retain its structural integrity, the lateral orifice facilitating alignment of the right-sided double lumen tube with the right upper lobe bronchus in various angular positions relative to a right main bronchus of the patient.

2. The right-sided double lumen tube of claim 1, wherein the lateral orifice has at most an angle of 180 degrees of 360 degrees of the circumference of the tube.

3. The right-sided double lumen tube of claim 1, wherein a portion of the first tube is adapted to be inserted in the right main bronchus and has a cross-section intersected by a coronal plane separating the cross section into anterior and posterior halves corresponding to ventral and dorsal portions of the patient's anatomy respectively, the lateral orifice centered about the coronal plane extending into both the anterior and posterior halves to accommodate encountered anatomical angular deviations of the right upper lobe bronchus in anterior and posterior directions relative to the coronal plane.

4. The right-sided double lumen tube of claim 3, wherein the cross-section is intersected by a transverse plane perpendicular to the coronal plane separating the cross section into upper and lower halves, the transverse plane intersected with the coronal plane forming four equal quadrants Q1, Q2, Q3 and Q4 in the cross-section, the anterior halve defined by Q1 and Q2, the posterior halve defined by Q3 and Q4, the upper halve defined by Q3 and Q1 and the lower halve defined by Q4 and Q2, the lateral orifice centered about the coronal plane extending into Q3 and Q1 to accommodate encountered anatomical angular deviations of the right upper lobe bronchus in anterior and posterior directions relative to the coronal plane.

5. The right-sided double lumen tube of claim 4, wherein the lateral orifice spans the entire upper halve of the cross section about the coronal plane.

6. The right-sided double lumen tube of claim 1, wherein the bronchial cuff is positioned adjacent the lateral orifice proximal thereto.

7. The right-sided double lumen tube of any one of claim 1, wherein the first tube has an axis and the lateral orifice has an axial length at least equal to a diameter of the right upper lobe bronchus.

8. A right-sided double lumen tube comprising at least one endobronchial tube adapted for insertion in a right main bronchus of a patient, the tube having a circumference defined by 360 degrees and a length defining a proximal and a distal end, the tube defining a lumen at the distal end and a lateral orifice defined about the circumference proximal the lumen, the lateral orifice being oversized relative to a right upper lobe bronchus of the patient to an extent accommodating encountered anatomical angular deviations of the right upper lobe bronchus in anterior and posterior directions relative to the right main bronchus.

9. The right-sided double lumen tube of claim 8, wherein the lateral orifice has an angular width of at least 80 degrees of the circumference of the endobronchial tube and at most an angular width permitting the tube to retain its structural integrity.

10. The right-sided double lumen tube of claim 8, wherein the lateral orifice has at most an angular width of 180 degrees of 360 degrees of the circumference of the tube.

11. The right-sided double lumen tube of claim 8, wherein the endobronchial tube has a cross-section intersected by a coronal plane separating the cross section into anterior and posterior halves corresponding to ventral and dorsal portions of the patient's anatomy respectively, the lateral orifice centered about the coronal plane extending into both the anterior and posterior halves to accommodate encountered anatomical angular deviations of the right upper lobe bronchus in anterior and posterior directions relative to the coronal plane.

12. The right-sided double lumen tube of claim 11, wherein the cross-section is intersected by a transverse plane perpendicular to the coronal plane separating the cross section into upper and lower halves, the transverse plane intersected with the coronal plane forming four equal quadrants Q1, Q2, Q3 and Q4 in the cross-section, the anterior halve defined by Q1 and Q2, the posterior halve defined by Q3 and Q4, the upper halve defined by Q3 and Q1 and the lower halve defined by Q4 and Q2, the lateral orifice centered about the coronal plane extending into Q3 and Q1 to accommodate encountered anatomical angular deviations of the right upper lobe bronchus in anterior and posterior directions relative to the coronal plane.

13. The right-sided double lumen tube of claim 12, wherein the lateral orifice spans the entire upper halve of the cross section about the coronal plane.

14. The right-sided double lumen tube of claim 8, further comprising a bronchial cuff enveloping the tube positioned adjacent the lateral orifice proximal thereto.

15. The right-sided double lumen tube of claim 8, wherein the endobronchial tube has an axis and the lateral orifice has an axial length at least equal to a diameter of the right upper lobe bronchus.

16. A right-sided double lumen tube comprising at least one tube having a circumference and an axis, the tube having a length defining a proximal tracheal end and a distal bronchial end for communicating fluid therebetween, the tube defining a lateral orifice about the circumference at the distal bronchial end, the lateral orifice adapted to face a right upper lobe bronchus of a patient and to optimize fluid communication therewith, the lateral orifice having an angular width of at least approximately 20% of the circumference of the tube and at most the angular width permitting the tube to retain its structural integrity, the lateral orifice having an axial length at least equal to a diameter of the right upper lobe bronchus.

17. The right-sided double lumen tube of claim 16, wherein the lateral orifice has an angular width of at most 50% of the circumference of the tube.

18. The right-sided double lumen tube of claim 16, wherein the tube has a cross-section intersected by a coronal plane separating the cross section into anterior and posterior halves corresponding to ventral and dorsal portions of the patient's anatomy respectively, the lateral orifice centered about the coronal plane extending into both the anterior and posterior halves to accommodate encountered anatomical angular deviations of the right upper lobe bronchus in anterior and posterior directions relative to the coronal plane.

19. The right-sided double lumen tube of claim 18, wherein the cross-section is intersected by a transverse plane perpendicular to the coronal plane separating the cross section into upper and lower halves, the transverse plane intersected with the coronal plane forming four equal quadrants Q1, Q2, Q3 and Q4 in the cross-section, the anterior halve defined by Q1 and Q2, the posterior halve defined by Q3 and Q4, the upper halve defined by Q3 and Q1 and the lower halve defined by Q4 and Q2, the lateral orifice centered about the coronal plane extending into Q3 and Q1 to accommodate encountered anatomical angular deviations of the right upper lobe bronchus in anterior and posterior directions relative to the coronal plane.

20. The right-sided double lumen tube of claim 19, wherein the lateral orifice spans the entire upper halve of the cross section about the coronal plane.

21. The right-sided double lumen tube of claim 16, further comprising a bronchial cuff enveloping the tube positioned adjacent the lateral orifice proximal thereto adapted to form an air-tight seal in a right main bronchus of the patient.

22. An endobronchial tube for insertion in a right main bronchus of a patient, comprising a tube body defining a lateral orifice adapted for fluid communication with a right upper lobe bronchus, the tube body being insertable in the right main bronchus in a first position in which the lateral orifice is generally facing the right upper lobe bronchus, the lateral orifice being sized independently of the right upper lobe bronchus position relative to the right main bronchus to accommodate, when the tube body is in the first position, anterior and posterior deviations of the right upper lobe bronchus relative to a coronal plane separating the patient's anatomy into ventral and dorsal portions.

23. The endobronchial tube of claim 22, wherein the tube has a cross-section intersected by the coronal plane separating the cross section into anterior and posterior halves corresponding to ventral and dorsal portions of the patient's anatomy respectively, the lateral orifice centered about the coronal plane extending into both the anterior and posterior halves to accommodate encountered anatomical angular deviations of the right upper lobe bronchus in anterior and posterior directions relative to the coronal plane.

24. The endobronchial tube of claim 23, wherein the cross-section is intersected by a transverse plane perpendicular to the coronal plane separating the cross section into upper and lower halves, the transverse plane intersected with the coronal plane forming four equal quadrants Q1, Q2, Q3 and Q4 in the cross-section, the anterior halve defined by Q1 and Q2, the posterior halve defined by Q3 and Q4, the upper halve defined by Q3 and Q1 and the lower halve defined by Q4 and Q2, the lateral orifice centered about the coronal plane extending into Q3 and Q1 to accommodate encountered anatomical angular deviations of the right upper lobe bronchus in anterior and posterior directions relative to the coronal plane.

25. The endobronchial tube of claim 24, wherein the lateral orifice spans the entire upper halve of the cross section about the coronal plane.

26. The endobronchial tube of claim 22, wherein the tube has a circumference and the lateral orifice has an oversized angular width at least 80 degrees of the circumference and at most an angular width permitting the tube to retain its structural integrity.

27. The endobronchial tube of claim 22, wherein the lateral orifice has an axial length at least equal to a diameter of the right upper lobe bronchus.
